(19) United States
(12) Patent Application Publication
(10) Pub. No.: US 2014/0277190 A1
(43) Pub. Date: Sep. 18, 2014

(54) COMPRESSION SCREW WITH VARIABLE PITCH THREAD

(71) Applicant: HOWMEDICA OSTEONICS CORP., Mahwah, NJ (US)

(72) Inventors: Roy Splieth, Central Valley, NY (US); Michael A. McGovern, Wyckoff, NJ (US); Andrew Nelson, Tinton Falls, NJ (US); Koustubh Rao, Hackensack, NJ (US)

(73) Assignee: HOWMEDICA OSTEONICS CORP., Mahwah, NJ (US)

(21) Appl. No.: 13/803,615

(22) Filed: Mar. 14, 2013

(51) Int. Cl. A61B 17/86

(52) U.S. Cl. CPC A61B 17/8605
USPC 606/308

(57) ABSTRACT

A bone screw apparatus is provided for threaded engagement with a bone and, optionally, a medical device such as a bone plate. The bone screw has a shaft having a first end, a second end, and a first thread having a first pitch. The screw further includes a head having a proximal end, a distal end adjacent the shaft first end, and a second thread having a second pitch. The second thread has a variable pitch that increases in a proximal direction, but never exceeds the magnitude of any portion of the first pitch. Upon threaded engagement of the head with bone or a medical device, a compression force is generated which continuously decreases as the screw is further inserted.

### BACKGROUND OF THE INVENTION

The present invention relates generally to the field of orthopedic compression screws, and more particularly to an orthopedic compression screw having a novel variable pitch thread on at least one portion of the screw.

Various types of fasteners have been used in fractured bone tissue and to engage surgical implants, plates, and other medical devices to bone tissue. Many existing bone screws include a threaded shaft portion adapted for engagement in bone and a head for coupling to a medical device such as the aforementioned implants and plates. In setting the bone fracture or implanting said prostheses, it is desirable to create a secure connection between the bone fragments or the implant and the bone by utilizing a compression screw.

Compression screws of the prior art include threaded heads to engage with another portion of bone or a threaded screw hole of the prosthetic device to be implanted. These screws typically utilize a thread having a continuously diminishing pitch from the distal to proximal ends of the screw such that there is a larger screw pitch along the shaft relative to the pitch of the head to create a “compression” force when the screw is inserted. The larger pitch along the shaft causes it to drive deeper than the head for every revolution. This “compression screw,” as it is known in the art, compresses the bone fragments together or the implant against the bone surface when the screw is inserted, thereby fastening the fragments or the implant securedly to the bone.

A problem with prior art compression screws is that the continuous variable pitch on the heads of the current designs either maintains or increases the compression force as the screw is inserted into the bone. As a result, the more proximal portions of the thread on the head, particularly if the head is tapered, are subject to greater localized stresses as the screw is advanced into the bone. These increasing forces can overly fatigue or deform the screw threads and, in some instances, can cause the thread running along the head to strip off the screw, potentially leaving small shards of metal inside a patient during implantation of the device.

Thus, there is a need for a compression screw that can limit or mitigate the effect of the forces on the screw threads and that can improve the ability of the screw threads to withstand such forces. In one particular embodiment, an improved screw can generate a continuous but decreasing compression force as the screw is advanced into the bone.

### BRIEF SUMMARY OF THE INVENTION

The present invention includes various embodiments of a screw having a threaded shaft and a threaded head, which can generate a compression force along the length of the screw when inserted into a bone. Generally, the screw creates a first compression rate when the shaft and the distal end are engaged with a material, and a second compression rate when the shaft and the proximal end are engaged with said material, the second compression rate being less than the first compression rate.

In one aspect of the present invention, a bone screw is provided having a shaft and a head. The shaft has a first end, a second end, and a first thread having a first pitch. The head has a proximal end, a distal end, and a second thread having a second pitch. The second pitch increases in a proximal direction, but is less than the first pitch at any point of the shaft.

In other embodiments, consecutive revolutions of the second thread can have an increased thickness at a base, a crest, or both, of the second thread in the proximal direction. The base, the crest, or both, of the second thread can increase in the proximal direction by a constant percentage at each consecutive revolution. The crest of the second thread can run parallel to the base of the second thread.

Consecutive revolutions of the second thread can have a constant thickness along a channel adjacent a base of the second thread in the proximal direction. The second thread can have first and second faces disposed at first and second constant angles, respectively, with respect to a surface of the head. The head can be conically tapered such that a minor diameter of the head increases in the proximal direction, and/or a major diameter of the head defined by the second thread increases in the proximal direction.

The shaft can have a third thread formed thereon and diametrically opposed to the first thread. The head can have a third thread formed thereon and diametrically opposed to the second thread. The elongate shaft can be conically tapered such that a minor diameter of the shaft and/or a major diameter of the shaft defined by the first thread decreases in a direction extending from the first end to the second end.

The first thread can have a constant pitch. A minor diameter of the shaft can be constant. The first pitch can have a constant value ranging between about 1.5 to 3.5 mm and the second pitch can have a maximum value ranging between about 0.5 to 1.0 mm, though other values for both pitches are possible. A channel defined by the second thread can be defined by a constant cross section.

In another embodiment, the first thread, the second thread, or both the first and second threads can be constructed with a Unified Thread Standard profile or an Acme thread profile.

In still another embodiment, a kit can be provided including a screw according to the present invention and a baseplate. The baseplate can define an aperture and can include a single annular rib disposed in the aperture, with the rib defining an inner rib diameter. The head of the screw can have a major head diameter having a maximum value and the shaft can include a major shaft diameter defined by the first thread. The inner rib diameter can be greater than the major shaft diameter, but less than the maximum value of the major head diameter.

Embodiments of the present invention may be comprised at least in part of titanium, a titanium alloy, cobalt chrome, or a combination thereof.

### BRIEF DESCRIPTION OF THE DRAWINGS

FIG. 1 is a front view of a screw in accordance with an embodiment of the present invention.

FIG. 2 is a cross-sectional view of the screw shown in FIG. 1 with a baseplate.

FIG. 3 is a schematic cross-sectional view of relative pitch size of a screw head of the present invention

FIG. 4 is a front view of a portion of a screw in accordance with another embodiment of the present invention.

FIG. 5 is a front view of a portion of a screw in accordance with still another embodiment of the present invention.

### DETAILED DESCRIPTION

FIG. 1 illustrates a front view of a bone screw 10 according to an embodiment of the present invention having a shaft 20 and a head 30. Shaft 20 has a proximal first end 21, a distal second end 22, and a first thread 23 formed thereon. The first thread 23 has a first pitch 24, which in the embodiment shown in FIG. 1 is constant. As known in the art, the pitch is the distance between two consecutive threads. Head 30 has a proximal end 31, a distal end 32 adjacent to the first end 21 of the shaft 20, and a second thread 33 formed thereon. The second thread 33 is conically shaped and has a variable pitch 34 that increases in a proximal direction extending from the distal end 32 of the head 30 to the proximal end 31 of the head 30, but is never greater in magnitude than the magnitude of the first pitch 24 at any portion of the shaft 20. That is, the pitch of any portion of the second thread 33 is less than the pitch of any portion of the first thread 23 at any point. Screw 10 includes an additional portion of head 30 proximal of proximal end 31 that is unthreaded. The references herein to the “ends” of a shaft or head are not meant to limit the present disclosure to requiring any shaft, head, or other portion to being fully threaded. Rather, any portion or the full extent of a shaft or head can be threaded.

FIG. 2 illustrates screw 10 engaged within a bone plate 40 having a screw-hole 41 and an annular rib 42 disposed within screw-hole 41. As shown, the threaded portion of the head 30 is conically shaped to facilitate engagement with the threaded rib 42. That is, the major diameter of the head 30 defined by the second thread 33 has a maximum value, and the inner diameter of annular rib 42 is less than the maximum value of the major diameter of head 30. This means that annular rib 42 engages second thread 33 at some point along the length of head 30, whereupon the thread 33 is rotated to cut into the rib 42. This interaction between thread 33 and rib 42 causes deformation of rib 42 at two equal and opposite locations along the circumference of the head 30 based upon the configuration of thread 33 to have a dual-lead. The interaction may also cause thread 33 to deform to some degree, although its strength compared with that of rib 42 usually results in a majority or all of the deformation occurring within rib 42. As shown, the inner diameter of annular rib 42 may also be greater than the major diameter of shaft 20, though such relationship is not required.

When screw 10 is inserted through the screw-hole 41 with shaft 20 engaged into the bone and head 30 engaged with rib 42, the relatively greater first pitch 24 of the first thread 23 of the shaft 20 causes the relatively smaller second pitch 34 of the second thread 33 to lag behind continuously as the screw 10 is inserted, thus generating a continuous compression force between the bone plate 40 and the surface of the bone. However, because the second pitch 34 increases in magnitude in the proximal direction, the compression force generated continuously decreases as the screw 10 is inserted. That is, the maximum compression force that screw 10 can generate is achieved at the point at which distal end 32 of head 30, or the lead end of second thread 33, engages with rib 42. From that point on, the variable nature of second pitch 34 causes the compression force generated by screw 10 to lower in magnitude. As the pitch of any portion of the second thread 33 is always less than the pitch of any portion of the first thread 23 at any point along shaft 20, a compression force is always applied by screw 10 no matter the depth to which the screw 10 is inserted.

Further referencing FIG. 2, during insertion of screw 10, the first thread 23 of the shaft 20 generates cancellous bone compression in a radial direction along its length as it is inserted. The tapered minor diameter of the shaft 20 creates a wedge within the bone after complete insertion. Thus, as screw 10 is advanced into the bone, the longitudinal compression force generated from the difference between the first and second pitch 24, 34 secures the plate 40 to the bone while the radial cancellous bone compression generated from the first thread 23 secures the screw 10 within the bone.

The continuous application of, but simultaneous decrease in, compression force achieved by screw 10 improves over the construction and use of prior art compression screws by still providing compression without the disadvantage of subjecting the thread of the lagging portion of the screw to increasing and excessive shear forces. Such screw thread deformation forces are accounted for at least in part by the variable construction of second pitch 34 of head 30. By increasing the second pitch 34 in the proximal direction, but never allowing the second pitch 34 to exceed any portion of the first pitch 24, the compression force decreases as the screw 10 is inserted. That is, use of the screw 10 creates a first compression rate when the shaft 20 and the distal end 32 of the head 30 are engaged with a material, and a second compression rate less than the first compression rate when the proximal portion of the second thread 33 is threadedly engaged with said material, a bone plate 40, or other medical device. In one embodiment of a screw in accordance with the present invention, the first pitch 24 of the shaft 20 can have a constant value ranging between about 1.5 to 3.5 mm and the second pitch 34 of the head 30 is variable with a maximum value ranging between about 0.5 to 1.0 mm.

During insertion of screw 10, the torque applied by the user on screw 10 generates localized forces along a mating surface between the second thread 33 and annular rib 42. These forces are dependent on the length of distance between the location at which they are applied and the axis of the shaft along which the torque is applied, which generally coincides with the minor radius of the head 30 at said mating surface. A certain amount of force is required to deform the mating interface, usually rib 42, to create a secure connection between the second thread 33 and rib 42.

However, these forces also generate stress upon the thread 33 at the mating interface, with that stress exerted upon thread 33 in a way that can loosen or strop thread 33 from head 30 in some configurations. The stress is a function of the shear area of the thread, which is defined as the area of the thread 33 that contacts rib 42. This amounts to the area defined by a crest 332, a distal-facing face or flank 333, and a proximal-facing face or flank 334 at any particular location of the thread 33. The shear stress applied to the thread is a function of the localized force on the thread 33 at a location divided by the area of the thread 33 at that location. If the force applied exceeds a certain threshold defined by the ultimate tensile strength of the material of the screw 10, the second thread 33 will begin to strip. By increasing the area of the second thread 33 in a proximal direction, which is the direction at which localized forces are also increasing, the shear stress on the second thread 33 at more proximal locations can be controlled or alleviated without compromising the amount of force needed to deform rib 42 during its interaction with the second thread 33 at the mating interface. As the lengths of flanks 333 and 334 are usually constant throughout thread 33 (though they can be variable), increasing the area of thread 33 in a proximal direction can be done by increasing the length of crest 332 as the thread 33 winds up the head 30 in a proximal direction. This can be seen in FIG. 3 as the crest 332 of the upper (more proximal) thread 33 is larger in magnitude than the crest 332 of the lower (more distal) thread 33. Understanding this relationship of the configuration of thread 33 in light of the localized forces thereon, the crest 332 of thread can be configured to increase at a constant or variable rate in the proximal direction. The configurations of flanks 333 and 334 can also be altered, as they contributed to the surface area as well. For example, a constant increase in the length of crest 332 can be calculated to coincide with the increase of localized forces along thread 33 given a constant torque applied to screw 10. Also, by maintaining a larger first pitch at all points of the shaft 20, a compression force is continuously generated throughout insertion of screw 10.

FIG. 3 is a schematic cross-sectional view of the relative size of the second pitch 34 of screw 10, as the second thread 33 moves along a proximal direction 50. As shown, a cross-section of the second thread 33 has a base 331, crest 332, distal-facing face or flank 333, and proximal-facing face or flank 334. The base 331 is the portion of thread 33 adjacent the minor diameter of thread 33 of head 30. The crest 332 is the portion of thread 33 opposite the base 331 that defines the major diameter of thread 33 and also the outermost portion of thread 33. As the thread 33 winds around the head 30 in a proximal direction, the base 331 and crest 332 increase in size, but the proximal- and distal-facing flanks 333, 334 maintain the same angles with respect to the base 331 and crest 332 with bases 331 generally disposed on a surface of the head 30. In other words, consecutive revolutions of the second thread 33 have an increased thickness at the base 331 and crest 332 in the proximal direction 50 and accordingly, the second pitch 34 increases in the proximal direction 50 as well. Either or both of these thicknesses can increase in the proximal direction by a constant percentage at each consecutive revolution. One or more of the crests 332 can run parallel to one or more of the bases 331. It is noted that FIG. 3 appears to show the bases 331 aligned in a generally vertical orientation. This is illustrative only, and the relative configuration of the threads can be disposed on a head having a constant or tapered minor diameter, with the minor diameter being generally coincident with the bases 331.

In addition, a channel 335 defined between consecutive revolutions of the second thread 33 can be configured to maintain the same size and cross-sectional configuration along the entire length of the second thread 33. Thus, the second thread 33 increases in cross-sectional thickness at its base 331 and crest 332 in a proximal direction, thereby increasing the second pitch 34, while the cross-sectional area of the channel 335 remains the same. However, in alternate embodiments, channel 335 may increase or decrease in size in a proximal direction, so long as the second pitch 34 increases in a proximal direction.

This configuration of second thread 33 results in thread 33 being constructed of more material as it winds about head 30 in the proximal direction. This increase in material is another aspect of the present invention that accounts for the increase in screw thread deformation forces that bear upon thread 33 as screw 10 is inserted. The additional material of thread 33 in the proximal portions of thread 33, and in particular the greater length of base 331 which effectively anchors thread 33 to head 30 of screw 10, results in greater strength of thread 33 in opposition to shear forces. As force upon thread 33 increases during insertion, so too does the area at which base 331 of thread 33 to improve the ability of the proximal portions of thread 33 to withstand the increasing shear forces. While the material of thread 33 can be strengthened using surface treatments, such treatments are not required due to the variable configuration of thread 33.

FIGS. 4 and 5 illustrate alternate embodiments of screws 10a and 10b of the present invention, with like numerals referring to like elements. As shown, the head 30a, 30b may be substantially threaded along its entire length or partially threaded as screw 10 shown in the embodiment of FIGS. 1 and 2. In addition, as shown in the embodiment of FIG. 4, the screw 10b may have a positive stop 35b in the form of a proximal end having a diameter larger than that of any distal portion of the screw 10b. Either of these alternate embodiments 10a, 10b could be compatible with bone plate 40 of FIG. 2 through threaded engagement with the annular ribs 42 of any screw hole 41 in plate 40.

Screws in accordance with the present invention can be constructed with any particular thread profile, such as a Unified Thread Standard profile (60 degree thread angle) or an Acme thread profile (29 degree thread angle).

As to any of the above embodiments, it is envisioned that design and structural modifications may be made within the scope of the present invention to provide a screw that creates a similar compressive effect or is configured to withstand external force to the threads of the head in a similar way. For example, the present invention may include a dually threaded head and/or shaft, such that the screw incorporates a third thread diametrically opposed to either the first or second thread, or both. Also, the first thread 23, although shown with a constant pitch, may have a variable pitch as long as the first pitch 24 is greater than the second pitch 34 along the first thread's 23 entire length. Furthermore, the shaft 20 may be conically tapered along its major or minor diameters, or both, with the minor diameter being the diameter of the shaft 20 without the thread 23 disposed thereon and the major diameter of the shaft 20 defined by the outer diameter of the thread 23 thereon. The shaft 20 can alternatively be of a constant diameter or can have both increasing and decreasing diametrical portions. In addition, the conical shape of the head 30 may be created by tapering the major or minor diameter or both, with the minor diameter being the diameter of the head 30 without the thread disposed thereon and the major diameter of the head 30 defined by the outer diameter of the thread 33 thereon. Additionally, the head 30 may be cylindrically shaped at its minor diameter so long as it is configured to engage with a baseplate, if such engagement is desired. Also, the screw may be designed to be self-tapping or to be drilled into a pre-cut hole. The configuration of the present compression screw may be utilized in many orthopedic applications, such as in a bone screw with a cortical plate, a fixation screw with a bone fracture, a pedicle screw with a spinal system, a set screw used in a coupling element of a pedicle screw, or in any other application in which any amount of compression is desired during insertion of a screw. Although the present invention is described in the context of surgery, it is understood that the invention may be used for any material that is at least slightly elastically compressible, such as wood.

The screws of the present invention can be manufactured from any rigid biocompatible material, such as metals, polymers, ceramics, and alloys thereof. In particular embodiments, a screw can be manufactured from titanium, titanium alloys, or cobalt chrome. In addition, the screws can have coatings such as anodization type II or type III to provide increased hardness and/or lubricity.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A bone screw, comprising:
a shaft having a first end, a second end, and a first thread formed thereon, the first thread having a first pitch; and
a head having a proximal end, a distal end adjacent to the first end of the shaft, and a second thread formed thereon;
wherein the second thread has a variable pitch that increases in a proximal direction extending from the distal end of the head to the proximal end of the head; and
wherein the pitch of any portion of the second thread is less than the pitch of any portion of the first thread at any point.

2. The screw of claim 1, wherein consecutive revolutions of the second thread have an increased thickness at a base of the second thread in the proximal direction.

3. The screw of claim 2, wherein consecutive revolutions of the second thread have an increased thickness at a crest of the second thread in the proximal direction.

4. The screw of claim 2, wherein the base of the second thread increases in the proximal direction by a constant percentage at each consecutive revolution.

5. The screw of claim 3, wherein the crest of the second thread increases in the proximal direction by a constant percentage at each consecutive revolution

6. The screw of claim 3, wherein the crest of the second thread runs parallel to the base of the second thread.

7. The screw of claim 1, wherein consecutive revolutions of the second thread have a constant thickness along a channel adjacent a base of the second thread in the proximal direction.

8. The screw of claim 1, wherein the second thread has first and second faces disposed at first and second constant angles, respectively, with respect to a surface of the head.

9. The screw of claim 1, wherein the head is conically tapered such that a minor diameter of the head increases in the proximal direction.

10. The screw of claim 9, wherein the head is conically tapered such that a major diameter of the head defined by the second thread increases in the proximal direction.

11. The screw of claim 1, wherein the shaft has a third thread formed thereon and diametrically opposed to the first thread.

12. The screw of claim 1, wherein the head has a third thread formed thereon and diametrically opposed to the second thread.

13. The screw of claim 1, wherein the elongate shaft is conically tapered such that a minor diameter of the shaft decreases in a direction extending from the first end to the second end.

14. The screw of claim 1, wherein the elongate shaft is conically tapered such that a major diameter of the shaft defined by the first thread decreases in a direction extending from the first end to the second end.

15. The screw of claim 1, wherein the elongate shaft is conically tapered such that major and minor diameters of the shaft each decrease in a direction extending from the first end to the second end, the major diameter of the shaft being defined by the first thread.

16. The screw of claim 1, wherein the first thread has a constant pitch.

17. The screw of claim 1, wherein a minor diameter of the shaft is constant.

18. The screw of claim 1, wherein the first pitch has a constant value ranging between about 1.5 to 3.5 mm and the second pitch has a maximum value ranging between about 0.5 and 1.0 mm.

19. The screw of claim 1, wherein a channel defined by the second thread is defined by a constant cross section.

20. A kit comprising:
the screw according to claim 1; and
a baseplate defining an aperture and including a single annular rib disposed in the aperture, the rib defining an inner rib diameter,
wherein the head of the screw includes a major head diameter having a maximum value,
wherein the elongate shaft includes a major shaft diameter defined by the first thread, and
wherein the inner rib diameter is greater than the major shaft diameter, but less than the maximum value of the major head diameter.

21. The screw of claim 1, wherein use of the screw creates a first compression rate when the shaft and the distal end are engaged with a material, and a second compression rate when the shaft and the proximal end are engaged with said material, the second compression rate being less than the first compression rate.

22. The screw of claim 1, wherein the screw is comprised at least in part of titanium, a titanium alloy, cobalt chrome, or a combination thereof.

23. The screw of claim 1, wherein the first thread, the second thread, or both the first and second threads is constructed with a Unified Thread Standard profile or an Acme thread profile.
